(19) United States
(12) Patent Application Publication
(10) Pub. No.: US 2007/0185569 A1
(43) Pub. Date: Aug. 9, 2007

(54) Drug eluting stent coating with extended duration of drug release

(76) Inventor: SoonKap Hahn, San Clemente, CA (US)

Correspondence Address: MICHAEL E. KLICPERA, PO BOX 573, LA JOLLA, CA, 92038-0573, US

(21) Appl. No.: 11/348,947

(22) Filed: Feb. 6, 2006

(51) Int. Cl. A61F 2/06

(52) U.S. Cl. 623/1.46

(57) ABSTRACT

A stent having a drug eluting formulation has three components: 1) Anti-neointimal hyperplasia or anti-restenosis agent 2) Main polymer 3) Additive polymer
The anti-neointimal hyperplasia or anti-restenosis agent includes, but not limited to, Paclitaxel, Taxol, Rapamycin, Tacrolimus, Actinomycin D, Methotrexate, Doxorubicin, cyclophosphamide, and 5-fluorouracil, 6-mercapatopurine, 6-thioguanine, cytoxan, cyclosporine, cytarabinoside, cis-platin, chlorambucil, busulfan, and any other drug that can inhibit cell proliferation, and combinations thereof. The main polymer includes, but not limited to, polystyrene, parylene and polyurethane. The additive polymer includes, but not limited to, polyethylene glycol capped with diisocyanate moiety (NCO-PEG).

TABLE
Ratio between three components without solvent

| | % |
|---|---|
| Component | formulation |
| Agent | 1-10% |
| Main polymer | 80-98% |
| Additive | 1-19% |
| polymer | |

9.0 g of parylene, 0.6 g of tacrolimus, 0.4 g of NCO-PEG and 0.01 g of triethylene amine were dissolved in 90 g of tetrahydrofuran. The resulting mixture was heated at 40° C. for 30 minutes and cooled to room temperature. To the solution was added 0.1 g of pH 8.0 aqueous solution and mixed thoroughly. The resulting solution is applied to bare metal stents for coating.

### FIELD OF THE INVENTION

The present invention relates to an endovascular drug-delivery stent and to a method for treating restenosis.

### BACKGROUND OF THE INVENTION

Coronary and peripheral angioplasty is routinely performed to treat obstructive atherosclerotic lesions in the coronary and peripheral blood vessels. Following balloon dilation of these blood vessels, 30-40% of patients undergo restenosis.

Restenosis is the reclosure of a peripheral or coronary artery following trauma to that artery caused by efforts to open a stenosed portion of the artery, such as, for example, by balloon dilation, ablation, atherectomy or laser treatment of the artery. Restenosis is believed to be a natural healing reaction to the injury of the arterial wall. The healing reaction begins with the thrombotic mechanism at the site of the injury. The final result of the complex steps of the healing process can be intimal hyperplasia, the uncontrolled migration and proliferation of medial smooth muscle cells, combined with their extracellular matrix production, until the artery is again stenosed or occluded. Thus, restenosis is characterized by both elastic recoil or chronic constriction of the vessel in addition to abnormal cell proliferation.

Currently restenosis must be treated with subsequent angioplasty procedures. In an attempt to prevent restenosis, metallic intravascular stents have been permanently implanted in coronary or peripheral vessels. For example, U.S. Pat. No. 5,304,122 (Schwartz et al.) describe metal stents useful for treating restenosis after balloon angioplasty or other coronary interventional procedures. The stent is typically inserted by catheter into a vascular lumen and expanded into contact with the diseased portion of the arterial wall, thereby providing mechanical support for the lumen. However, it has been found that restenosis can still occur with such stents in place; likely, because although the stent prevents elastic recoil of the artery, it fails to prevent the cell proliferation which leads to intimal hyperplasia. In addition, the stent itself can cause undesirable local thrombosis. To address the problem of thrombosis, persons receiving stents also receive extensive systemic treatment with anticoagulant and antiplatelet drugs.

A stent is a type of endovascular implant, usually generally tubular in shape, typically having a metallic lattice, connected-wire tubular construction which is expandable to be permanently inserted into a blood vessel to provide mechanical support to the vessel and to maintain or re-establish a flow channel during or following angioplasty. The support structure of the stent is designed to prevent early collapse of a vessel that has been weakened and damaged by angioplasty. In addition, delivery of stents has been shown to prevent negative remodeling and spasm of the vessel while healing of the damaged vessel wall proceeds over a period of months.

During the healing process, inflammation caused by angioplasty and stent implant injury often causes smooth muscle cell proliferation and regrowth inside the stent, thus partially closing the flow channel, and thereby reducing or eliminating the beneficial effect of the angioplasty/stenting procedure. This process is called restenosis. Blood clots may also form inside of the newly implanted stent due to the thrombotic nature of the stent surfaces, even when biocompatible materials are used to form the stent.

While large blood clots may not form during the angioplasty procedure itself or immediately post-procedure due to the current practice of injecting powerful anti-platelet drugs into the blood circulation, some thrombosis is always present, at least on a microscopic level on stent surfaces, and it is thought to play a significant role in the early stages of restenosis by establishing a biocompatible matrix on the surfaces of the stent whereupon smooth muscle cells may subsequently attach and multiply.

Stent coatings are known which contain bioactive agents that are designed to reduce or eliminate thrombosis or restenosis. Such bioactive agents may be dispersed or dissolved in either a bio-durable or bio-erodable polymer matrix that is attached to the surface of the stent wires prior to implant. After implantation, the bioactive agent diffuses out of the polymer matrix and preferably into the surrounding tissue over a period lasting at least 4 weeks, and in some cases up to 1 year or longer, ideally matching the time course of restenosis, smooth muscle cell proliferation, thrombosis or a combination thereof.

If the polymer is biodegradable, in addition to release of the drug through the process of diffusion, the bioactive agent may also be released as the polymer degrades or dissolves, making the agent more readily available to the surrounding tissue environment. When biodegradeable polymers are used as drug delivery coatings, porosity is variously claimed to aid tissue ingrowth, make the erosion of the polymer more predictable, or to regulate or enhance the rate of drug release, as, for example, as disclosed in U.S. Pat. Nos. 6,099,562, 5,873,904, 5,342,348, 5,873,904, 5,707,385, 5,824,048, 5,527,337, 5,306,286, and 6,013,853.

Heparin, as well as other anti-platelet or anti-thrombolytic surface coatings, are known which are chemically bound to the surface of the stent to reduce thrombosis. A heparinized surface is known to interfere with the blood-clotting cascade in humans, preventing attachment of platelets (a precursor to thrombin)on the stent surface. Stents have been described which include both a heparin surface and an active agent stored inside of a coating (see U.S. Pat. Nos. 6,231,600 and 5,288,711, for example).

A variety of agents specifically claimed to inhibit smooth muscle-cell proliferation, and thus inhibit restenosis, have been proposed for release from endovascular stents. As an example, some stents are coated with taxol or paclitaxel, a cytotoxic agent thought to be the active ingredient in the agent taxol.

In addition, rapamycin, an immunosuppressant reported to suppress both smooth muscle cell and endothelial cell growth, has been shown to have improved effectiveness against restenosis, when delivered from a polymer coating on a stent.

Ideally, a compound selected for inhibiting restenosis, by drug release from a stent, should have three properties. First, because the stent should have a low profile, meaning a thin drug/coating matrix. Second, the drug/coating matrix should be sufficiently active to produce a continuous therapeutic dose for a minimum period of 2-10 weeks when released from a polymer coating. Third, the compound should be effective in inhibiting smooth muscle cell proliferation.

### SUMMARY OF THE INVENTION

The drug eluting stent uses a stent which is coated with an anti-neointimal hyperplasia agent and a polymer, aiming a local, sustained release of the agent. The current drug eluting stents approved by the Food and Drug Administration currently use coatings in which agents are only physically entrapped. This type of coating formulations releases agents only through a diffusion control with a limited duration of drug release.

A new drug eluting formulation enabling the physical entrapment and chemical binding of agents is designed in this invention disclosure. This new formulation extends the duration of drug release beyond currently commercially available formulations.

The new drug eluting formulation has three components: 1) Anti-neointimal hyperplasia agent 2) Main polymer 3) Additive polymer

The anti-neointimal hyperplasia agent includes, but not limited to, taxol, sirolimus, tacrolimus, methotrexate and cyclosporine. The main polymer includes, but not limited to, polystyrene, parylene and polyurethane. The additive polymer includes, but not limited to, polyethylene glycol capped with diisocyanate moiety (NCO-PEG). Three components in organic solvent such as tetrahydrofuran or chloroform, but not limited to, with a different ratio (shown in the following table) can be formulated and applied to bare metal stents as a coating formulation. As an option, a small amount of water (less than 1% of total volume; basic solution with pH higher than 7.4) and/or catalytic amount of base such as triethylamine, but not limited to, can be added into the above organic solution formulation to facilitate reactions between isocyanate and hydroxyl function of the agent. The urethane bond between the agent and isocyanate of NCO-PEG is stable under dry condition but is labile in moisture environment over time.

Accordingly, one of the objects of the present invention is to provide a drug eluting stent that utilizes a coating/drug matrix that extends the duration of drug release.

Another object of the present invention is to provide methods for treating coronary and peripheral vascular diseases, particularly restenosis and vein by-pass grafts, using the drug coated stents which have the characteristic of an extended duration of drug release.

### BRIEF DESCRIPTION OF THE DRAWINGS

FIG. 1 illustrates an endovascular stent having a metal-filament body, and formed in accordance with the present invention, showing the stent in its contracted configuration.

FIG. 2 illustrates an endovascular stent having a metal-filament body, and formed in accordance with the present invention, showing the stent in its expanded configuration.

FIG. 3 is an enlarged cross-sectional view of a coated metal filament in the stent of FIG. 1 or FIG. 2.

FIG. 4 is a cross-sectional view of an example stent with the present invention coating/drug that is deployed at a vascular site and partly embedded within the vascular tissue.

### DESCRIPTION OF THE PREFERRED EMBODIMENTS

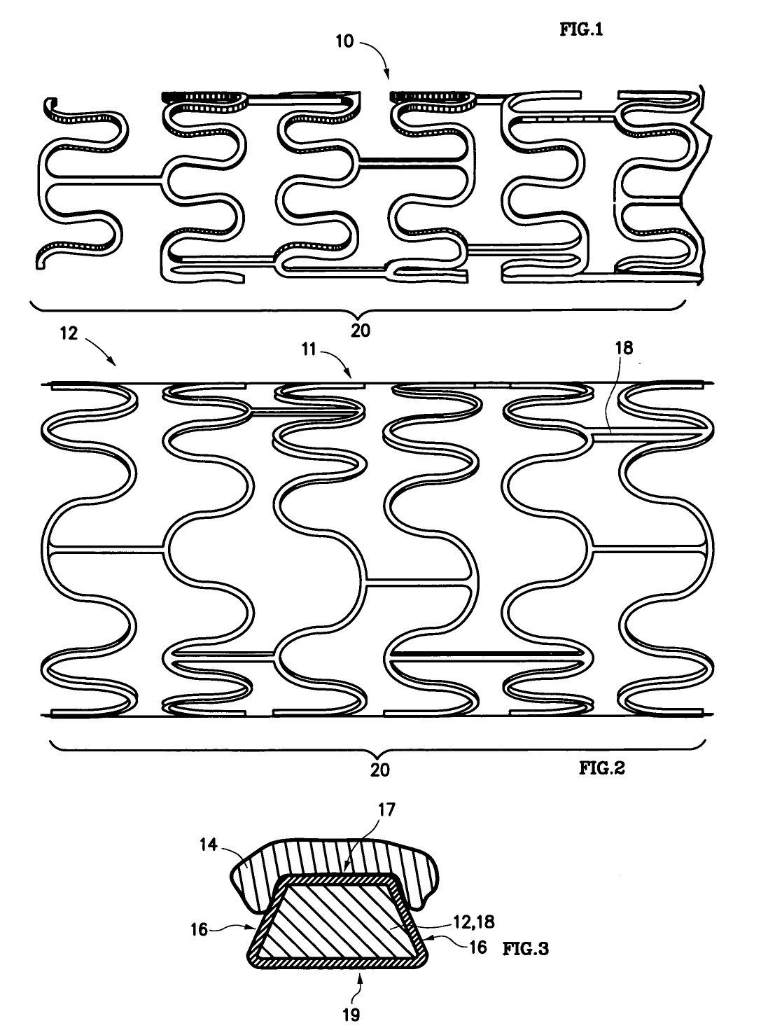

FIGS. 1 and 2 show a stent 10 constructed in accordance with the invention, in the stent's contracted and expanded states, respectively. The stent includes a structural member 12 and an outer coating 14 for holding and slowly releasing an anti-restenosis drug.

A. Stent Body

As shown for the purpose of an example stent 10, the stent body 20 of FIGS. 1 and 2 is formed of a plurality of linked tubular members by filaments, such as member 16. Each member is an expandable zig-zag, sawtooth, or sinusoidal wave structure. The members are linked by axial links, such as links 18 joining the peaks and troughs of adjacent members. As can be appreciated, this construction allows the stent to be expanded from a contracted condition, shown in FIG. 1, to an expanded condition, shown in FIG. 2, with little or no change in the length of the stent. The example stent has a contracted configuration diameter (FIG. 1) of between 0.5-2.0 mm, and more preferably between 0.70 to 1.50 mm, with a length of between 5-100 mm, and more preferably between 10-30 mm. In its expanded state, as shown in FIG. 2, the example stent diameter is several times larger than that of the stent in its contracted state. Thus, a stent with a contracted diameter of between 0.70 to 1.50 mm may expand radially to a selected expanded state of between 2-6 mm. When the typical stent is expanded and deployed with a vascular segment, some, if not most, of the structural members become engaged to the vessel wall and some, if not most of the structural members become partially or substantially embedded into the vessel wall 22.

Stents typically have a stent-body 20 architecture of linked, expandable structural 12 and linking members 18 is shown as an example stent. Alternatively, the structural member in the stent may have a continuous helical ribbon construction, that is, where the stent body is formed of a single continuous ribbon-like coil. There are many stent designs that the Applicant believes can be used with the present invention polymer/drug coating 14. The basic requirement of the stent body is that it be expandable, upon deployment at a vascular injury site, and that it is suitable for having a drug-containing coating on its outer surface, for slowly delivering the drug or therapeutic agent contained in the coating into the vessel wall (i.e. medial, adventitial, and endothelial layers of tissue) of the vascular site intended for intervention.

B. Stent Coatings

According to an important feature of the invention, the stent structural members 12 and linking member 18 are coated with a novel drug-release coating 14 composed of a multiple polymer matrix and an anti-neointimal hyperplasia or anti-restenosis compound (bioactive compound) that is incorporated within the multiple polymer matrix and designed to release the bioactive compound from the stent over an extended period of time.

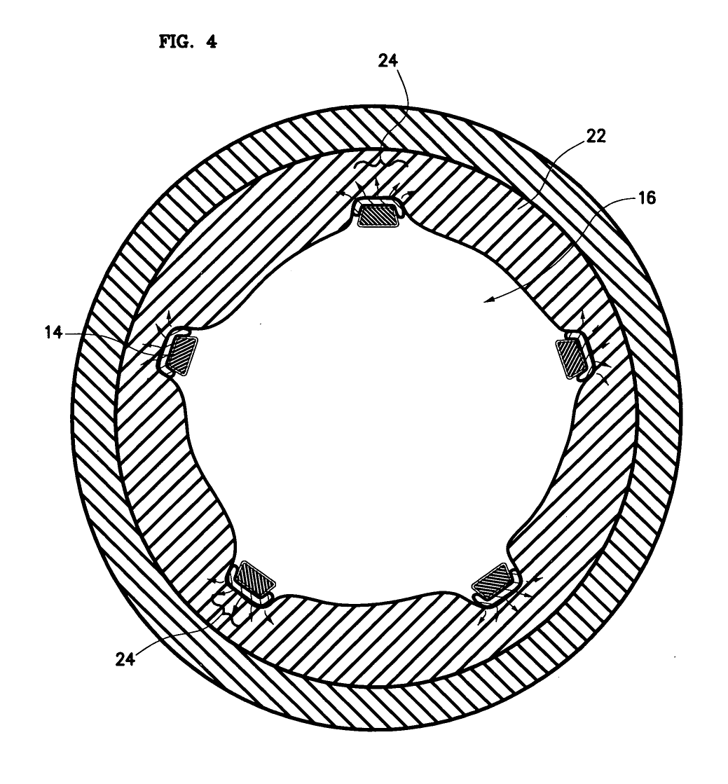

FIG. 3 shows, in enlarged sectional view, structural member 12 or linking member 18 of a typical stent having a drug/coating 14 that covers the structural 12 or linking 18 member substantially on the outside surface 17. Typical methods for coating a stent (dipping, spraying, etc.) may be less accurate than desired, or it may be intended that the polymer/drug coating 14 may also cover partially or substantially one or both sides 16 of the structural member 12. Alternately, the drug/coating 14 can cover all sides, that is, the side forming the outer surface 17 of the stent body, the bottom (the side forming the inside surface 19 of the stent) and the opposing sides 16. The trapezoidal shape of the structural member 12 or linking member 18 in FIG. 3 is only an example. Typical stents that could use the present invention coating include other shapes or configurations, e.g. square, round, oval, rectangle. The shape or configuration of the stent design is not particularly important to the present invention except that the design should have an outside surface (vessel wall facing) that can be coated with the novel present invention coating 14 that should become substantially engaged with the vessel wall upon deployment.

As will be discussed further below, the present invention coating 14 has a thickness typically between 3 and 30 microns, depending on the nature of the multiple polymer matrix material forming the coating and the relative amounts of polymer matrix and active compound. Ideally, the coating is made as thin as possible to minimize the stent profile in the vessel at the injury site.

It is desirable that the coating should also be relatively uniform in thickness across the outer surfaces 17, to promote even distribution and delivery of the released drug or therapeutic agent to the target site. Methods for producing a relatively even coating thickness on stent structural 12 and linking members include technology know to those skilled in the art.

The novel drug eluting formulation has three components: 1) Anti-neointimal hyperplasia or anti-restenosis agent; 2) a first main polymer; and 3) a second additive polymer.

The anti-neointimal hyperplasia or anti-restenosis agent includes, but are not limited to, Paclitaxel, Taxol, Rapamycin, Tacrolimus, Actinomycin D, Methotrexate, Doxorubicin, cyclophosphamide, and 5-fluorouracil, 6-mercapatopurine, 6-thioguanine, cytoxan, cyclosporine, cytarabinoside, cis-platin, chlorambucil, busulfan, and any other drug that can inhibit cell proliferation, and any combinations thereof.

The first main polymer includes, but not limited to, polystyrene, parylene and polyurethane.

The second additive polymer includes, but not limited to, polyethylene glycol capped with diisocyanate moiety (NCO-PEG).

To fabricate the coating matrix 14, all three components are dissolved in an organic solvent such as tetrahydrofuran or chloroform, but not limited to, with a different ratio (as shown in the Table 1 provided below) that can be formulated and applied to various bare metal stents as a coating formulation. As an option, a small amount of water (less than 1% of total volume; basic solution with pH higher than 7.4) and/or catalytic amount of base such as triethylamine, but not limited to, can be added into the above organic solution formulation to facilitate reactions between isocyanate and hydroxyl function of the agent. The urethane bond between the agent and isocyanate of NCO-PEG is stable under dry condition but is labile in moisture environment over time.

The mole amount of the agent is in excess of the mole amount of isocyanate of NCO-PEG. This ratio ensures a majority of the agent is physically entrapped for initial release.

TABLE 1
Ratio between three components without solvent

| | % |
|---|---|
| Component | formulation |
| Agent | 1-10% |
| Main polymer | 80-98% |
| Additive | 1-19% |
| polymer | |

### EXAMPLE 1

9.0 g of parylene, 0.6 g of tacrolimus, 0.4 g of NCO-PEG and 0.01 g of triethylene amine were dissolved in 90 g of tetrahydrofuran. The resulting mixture was heated at 40° C. for 30 minutes and cooled to room temperature. To the solution was added 0.1 g of pH 8.0 aqueous solution and mixed thoroughly. The resulting solution is applied to bare metal stents for coating.

1. A stent for placement at a vascular site for inhibiting restenosis at said injury site, comprising:
a stent body formed from a plurality of filaments;
a drug-release coating matrix composed of: (i) 80-98% weight percent polystyrene first main polymer; (ii) 1-19 weight percent of a second polyethylene glycol capped with diisocyante moiety, and (iii) an anti-neoinitimal or anti-restenosis agent;
said stent being expandable from a contracted condition in which the stent can be delivered to a vascular injury site via a catheter, and an expanded condition in which the stent coating can be placed in contact with the vessel at the injury site; and
said coating being effective to release said anti-neoinitimal or anti-restenosis agent of the matrix over an extended period of time.

2. A stent as recited in claim 1, wherein said anti-neoinitimal or anti-restenosis agent is Paclitaxel, Taxol, Rapamycin, Tacrolimus, Actinomycin D, Methotrexate, Doxorubicin, cyclophosphamide, and 5-fluorouracil, 6-mercapatopurine, 6-thioguanine, cytoxan, cyclosporine, cytarabinoside, cis-platin, chlorambucil, busulfan, and any other drug that can inhibit cell proliferation, and combinations thereof.

3. A stent for placement at a vascular site for inhibiting restenosis at said injury site, comprising:
a stent body formed from a plurality of filaments;
a drug-release coating matrix composed of: (i) 80-98% weight percent parylene first main polymer; (ii) 1-19 weight percent of a second polyethylene glycol capped with diisocyante moiety, and (iii) an anti-neoinitimal or anti-restenosis agent;
said stent being expandable from a contracted condition in which the stent can be delivered to a vascular injury site via a catheter, and an expanded condition in which the stent coating can be placed in contact with the vessel at the injury site; and
said coating being effective to release said anti-neoinitimal or anti-restenosis agent of the matrix over an extended period of time.

4. A stent as recited in claim 3, wherein said anti-neoinitimal or anti-restenosis agent is Paclitaxel, Taxol, Rapamycin, Tacrolimus, Actinomycin D, Methotrexate, Doxorubicin, cyclophosphamide, and 5-fluorouracil, 6-mercapatopurine, 6-thioguanine, cytoxan, cyclosporine, cytarabinoside, cis-platin, chlorambucil, busulfan, and any other drug that can inhibit cell proliferation, and combinations thereof.

5. A stent for placement at a vascular site for inhibiting restenosis at said injury site, comprising:
a stent body formed from a plurality of filaments,
a drug-release coating matrix composed of: (i) 80-98% weight percent polyurethane first main polymer; (ii) 1-19 weight percent of a second polyethylene glycol capped with diisocyante moiety, and (iii) an anti-neoinitimal or anti-restenosis agent;
said stent being expandable from a contracted condition in which the stent can be delivered to a vascular injury site via a catheter, and an expanded condition in which the stent coating can be placed in contact with the vessel at the injury site; and
said coating being effective to release said anti-neoinitimal or anti-restenosis agent of the matrix over an extended period of time.

6. A stent as recited in claim 5, wherein said anti-neoinitimal or anti-restenosis agent is Paclitaxel, Taxol, Rapamycin, Tacrolimus, Actinomycin D, Methotrexate, Doxorubicin, cyclophosphamide, and 5-fluorouracil, 6-mercapatopurine, 6-thioguanine, cytoxan, cyclosporine, cytarabinoside, cis-platin, chlorambucil, busulfan, and any other drug that can inhibit cell proliferation, and combinations thereof.

7. A method for treating vascular injury site, comprising:
delivering to the vascular injury site, an stent comprising;
a stent body formed from a plurality of structural member,
a drug-release coating matrix composed of: (i) 80-98% weight percent polystyrene, parylene or urethane first main polymer; (ii) 1-19 weight percent of a second polyethylene glycol capped with diisocyante moiety, and (iii) an anti-neoinitimal or anti-restenosis agent,
said stent being expandable from a contracted condition in which the stent can be delivered to a vascular injury site via a catheter, and an expanded condition in which the stent coating can be placed in contact with the vessel at the injury site, and
said coating being effective to release said anti-neoinitimal or anti-restenosis agent of the matrix over an extended period of time,
expanding the stent at the vascular injury site, to bring the stent coating in contact with the vessel at the injury site.
